(19) United States
(12) Patent Application Publication
(10) Pub. No.: US 2008/0221519 A1
(43) Pub. Date: Sep. 11, 2008

(54) System for Guiding a Probe Over the Surface of the Skin of a Patient or an Animal

(75) Inventors: Carol Schwach, Ribeauville (FR); Sieglinde Neerken, Eindhoven (NL); Gerhardus Wilhelmus Lucassen, Eindhoven (NL); Marion Geerligs, Eindhoven (NL); Frederikus Johannes Maria De Vreede, Eindhoven (NL); Robertus Hekkenberg, Eindhoven (NL); Nicole Leonarda Wilhelmina Eikelenberg, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/916,823

(22) PCT Filed: Jun. 6, 2006
(86) PCT No.: PCT/IB06/51797

§ 371 Date: Dec. 7, 2007

(30) Foreign Application Priority Data
Jun. 10, 2005 (EP) 05105115.9

(51) Int. Cl. A61M 5/42

(52) U.S. Cl. 604/116

(57) ABSTRACT

The invention relates to a system for guiding a probe over the surface of the skin of a patient. Performing measurements with ultrasound techniques for small structures such as blood vessels need well-defined spatial relationship between the measuring probe and the tissue which has to be examined. The invention seeks to ensure a movement of the measuring probe in a controlled and precise way over the skin. Therefore, a system 1 is suggested which comprises a probe holder 2 to which a probe 4 is rigidly attachable. The probe holder 2 is moveable along at least one rail 5, 5′. The rail can be wrapped around the patient 3 or a body part 7 of the patient or the animal.

The invention relates to the medical diagnosis of the skin and of tissue and structures below the skin, and in particular to the field of cannulation, hence to the insertion of a cannula or needle into the vascular system of a person or an animal.

For medical diagnosis of the skin or of structures below the skin with ultrasound systems or the like a measuring probe has to be positioned above the skin such that the ultrasound waves can penetrate the patient's body. The ultrasound may then be used to generate two-dimensional (2D) or three-dimensional (3D) images of the scanned body portions yielding images of e.g. blood vessels, a kidney or a liver.

In particular when imaging of smaller objects such as blood vessels is to be performed a movement of the probe relative to the scanned object becomes more critical. Such a movement decreases the effective spatial resolution of the images and should thus be avoided. However, moving and holding a probe in place by hand and optimizing its position is a difficult task, especially when additional tasks have to be carried out.

In order to avoid such a movement the patient is in many cases not allowed to move or even has to hold his breath. Results are, however, not satisfactory when the patient still moves too much, which is particularly the case with small children.

U.S. Pat. No. 6,478,740 B2 discloses an ultrasound imaging system with a hand-held satellite. The satellite comprises a motorized transducer which moves across the skin and which can be used to localize a blood vessel.

U.S. Pat. No. 6,530,886 B1 discloses an apparatus for measuring subcutaneous fat with ultrasound. An ultrasound probe is slidably mounted on an apparatus which is fastened to the patient's body by means of a belt.

It is an object of the invention to avoid the above-mentioned problems of the prior art and to provide a system for guiding a probe over the skin of a patient by which the movement of the probe can be controlled in a very precise way.

This object and other objects are solved by the features of the independent claims. Further embodiments of the invention are described by the features of the dependent claims. It should be emphasized that any reference signs in the claims shall not be construed as limiting the scope of the invention.

The system comprises a probe holder to which a probe can be rigidly attached. Further, at least one flexible rail is used which can be wrapped around the patient or a part of the body of the patient, where said probe holder is moveably mounted or is moveably mountable on said rail.

The term “patient” used in this description should be understood to include human beings as well as animals.

A probe can be fastened to the probe holder by conventional fastening means such as screws, snap-on mountings or the like. In this way a fixed spatial relationship exists between the probe holder and the probe. The rail is wrapped around the patient, and is then firmly attached to him. Furthermore, due to the attachment of the probe holder to the at least one rail there is no unwanted movement of the probe relative to the patient's body. As the probe holder is moveably mounted or is moveably mountable on the rail, a precise movement along the rail is possible to search for an optimal position of the probe.

The solution presented in the last paragraph allows a very precise movement of the probe holder, and thus of the probe, either manually, semi-automatically or automatically. To improve this precision even more the position of the probe holder can be fastened to the rail. Prior to this fastening the position of the rails relative to the patient's body can be easily changed in order to optimize the measurement position of the probe.

In the simplest case the probe holder is just a plate which is moveably mounted or is moveably mountable on the at least one rail, so that the probe holder can easily translate along the rail.

The at least one rail comprises a flexible material so that it can be wrapped around the patient or a body organ of the patient. It may thus be wrapped around a leg, a hand, an arm or other parts of the body.

The probe holder may accommodate at least a portion of the upper part of the rail, for example by means of a recess. If the material of the holder in the vicinity of the recess is rigid, the end of the rail can be inserted into the recess. If the material is elastic, the additional possibility exists of clicking the probe holder onto the rails. The rail then snaps into the recess of the probe holder and establishes the moveable relationship between the probe holder and the rail.

In a preferred embodiment the system comprises at least two flexible rails which are arranged substantially parallel to each other, and the probe is moveably mounted between said rails. This provides the advantage that the system is more securely attached to the body site. In particular, a rotational movement of the probe holder around an axis perpendicular to the rail is prohibited by this choice. Furthermore, a bigger probe can be attached more securely to the probe holder as a solution with two rails minimizes shear forces on the rails and/or on the above-mentioned recess for housing the rails.

In a further embodiment the rails are mounted onto a strap which is attachable to the patient. For that purpose the strap is made of a flexible material, for example a polymer, so that the flexible strap can be wrapped around the patient or the animal or a body organ of the patient. The strap facilitates and speeds up wearing a system having two or more rails as only a single strap has to be attached to the body site instead of a multitude of parts. Furthermore, wearing a strap is more convenient than wearing single rails. The straps thus allow a more convenient and faster use of the system.

In a further embodiment the probe holder is moveable substantially parallel as well as substantially vertical to the at least one rail. As explained above, the probe holder may have a recess which accommodates the upper part of the rail so that the probe holder can move along the rail. Furthermore, the rail itself can be moveable in a direction substantially vertical to the rails. For this purpose the at least one rail can be slidably mounted on the strap, for example by mounting the rail on a plate, where the plate is mounted on a second rail which shows an orientation perpendicular to the orientation of the first rail. This embodiment allows a two-dimensional movement of the probe over the skin.

In a further embodiment the strap comprises a Velcro fastener. The Velcro fastener helps to adjust the length of the strength and ensures that the strap fits tightly to the body site. Furthermore, it enables to compensate different body sizes as well as different strap sizes which are needed for different parts of the body. In the latter case, however, different straps for different body sites are preferred.

In a further embodiment the system comprises means for securing the position of the probe holder relative to the at least one rail. The securing means might be an element which is pressed against a rail as a brake. When the probe is already in the optimal position for performing a measurement, the position of the probe relative to the skin is fixed with the above-mentioned brake. The securing means has the advantage that the position need not be fixed manually enabling a hand-free operation such that the medical staff which uses the system can conveniently execute other tasks.

In another aspect of the invention the system accommodates a puncture system for inserting a cannula or a needle into a blood vessel of the patient. The probe holder then contains a probe adapted for this purpose, while the probe applies techniques such as Near infrared imaging, Optical Coherence Tomography, Photo Acoustic Imaging or Ultrasound Techniques. Furthermore, techniques based on Doppler signals can be used, for example Doppler Ultrasound or Doppler Optical Coherence Tomography. Also, combinations of Doppler-based and imaging-based signal acquisition techniques might be implemented.

An embodiment of a guiding system which cooperates with a puncture system as it is described in the last paragraph offers the possibility of performing blood withdrawals, infusions, catheter insertion, dialysis applications or the like. In comparison with an operation without this system these tasks can be performed with an increased safety for the patient and with an increased likelihood that the blood vessel is really punctured. This is particularly true in the case of less experienced medical staff. As a consequence, this group of persons can be appointed to carry out these tasks at reduced costs. Furthermore, the increased safety and comfort allows that the patient uses the system at home which, together with appropriate blood vessel parameter analysis tools, enables more frequent measurements.

The puncture system allows the operator to work manually, semi-automatically or even automatically when carrying out one of the above-mentioned tasks.

In another embodiment of the present invention the system comprises actuation means which are adapted to move the probe holder over the skin of the patient. This is preferably done in response to an output of the puncture system. The movement is then possible in directions parallel and/or perpendicular to the rail. In this way the system is able to autonomously determine an optimal position for a measurement by the probe, and to determine an optimal position for the insertion of a cannula. This provides a higher degree of automation and an increased comfort for the operator.

In another embodiment the puncture system comprises location determining means for determining at least one location of the blood vessel, and processing means for determining a puncture location of the blood vessel in response to an output of the location determining means. The location determining means carry out measurements by using the probe mentioned above, and the processing means, e.g. a computational entity and software, analyse the measurement values accordingly. The processing means may visualize the result on a screen, for example as a 2D image or a 3D image showing a blood vessel.

Typically, the location determining means are adapted to provide a plurality of geometric data of the blood vessel. This allows the determination of parameters such as blood vessel diameter, blood vessel size as well as a depth under the skin. Further, the location determining means effectively provide determination of the blood vessel's course. An effective use of such geometric and location information of the blood vessel allows the determination of an optimal puncture location with a high accuracy and reliability that finally allows to minimize a danger of injury of a vessel wall. Consequently, the generation or severity of bleeding, haematoma or inflammation can be minimized. Also by effective usage of obtained geometric and location data of the blood vessel, multiple attempts for a needle or cannula insertion can be prevented, because the reliable and accurate inspection of the blood vessel prior to insertion of the needle or cannula nearly guarantees that the needle or cannula can be correctly inserted or introduced into the vascular system with a single attempt. In particular, in emergency situations it is obvious, that this guided puncture is highly advantageous compared to an entirely manual cannulation.

When the system is equipped with a puncture system as described above this puncture system may comprise a light source and projection means associated with the light source. In this case the projection means are adapted to indicate the puncture location and the course of the blood vessel on the skin of the patient by light. This embodiment is useful in the case of a manual or a semi-automatic cannulation, as the positioning of the cannula onto the skin and the alignment of the cannula after the positioning is guided by the light.

As an example, the puncture location can be marked by a cross or another figure, and the course of the vessel can be visualized by an arrow or a line. Furthermore, the angle of the cannula with respect to the skin can be indicated as well. The projection means may include a tiltable mirror reflecting the light emitted by the light source. The light can be laser light, e.g. a laser pointer, or the light of a light-emitting diode. The light is preferably green light, as green light is easily visible on all types of skin, i.e. on light skin as well as on dark skin.

According to a further embodiment of the invention, the location determining means are further adapted to track the location of the needle's or cannula's distal end during insertion of the needle or cannula. The puncture system further has control means for controlling the movement of the needle or cannula in response to the tracking of the needle's or cannula's distal end. In this way, the puncture system is provided with a feedback allowing to monitor and to check whether the distal end of the needle or cannula is correctly inserted. This functionality effectively represents a safety mechanism of the puncture system and helps to prevent that despite of an accurate inspection of the blood vessel the cannula might be incorrectly introduced, which may have serious consequences for the patient's health.

Typically, the location determining means provide course and location determination of the blood vessel as well as tracking of the needle's or cannula's distal end at a sufficient repetition rate that allows fast reaction in case the cannula introduction deviates from a predetermined path or schedule. Also, the location determining means allow to check whether the distal end of the needle or cannula has been inserted correctly into the person's vascular system. Hence, the location determining means not only provide a control mechanism during needle or cannula insertion but also allow to check the final position of the needle or cannula after the intravascular insertion has been terminated.

Instead of tracking the needle's or cannula's distal end it is also possible to monitor and follow the position, size or movement of the blood vessel during insertion. In principle this should also provide enough information and is a somewhat simpler solution. If it is known where the needle or cannula has to end up and if the insertion parameters have been determined, the location and size of the blood vessel can be monitored during insertion. The insertion, however, goes wrong if the blood vessel does not stay in place.

The blood vessel identification means are adapted to identify whether a blood vessel is an artery or a vein. With this additional functionality the cannula insertion system is safer to use as many applications require a puncturing of the correct blood vessel type. This makes it possible that less experienced medical staff use the cannula insertion system, an aspect which saves money in the expensive health system. It is even conceivable that patients having no medical knowledge use the cannula insertion system under the supervision of medical staff. Procedures like blood withdrawal would then become particularly easy.

A first possibility of identifying the blood vessel type consists in applying conventional Doppler techniques with or without imaging, in particular with an ultrasonic or optical Doppler system. In this case an ultrasound or optical signal is coupled to the tissue containing the blood vessel and then absorbed by particles in the blood. The ultrasound or optical energy is then emitted by the particles and detected by a sensor as a Doppler signal. Blood flowing away from the sensor emits ultrasound or optical waves having a frequency that is lower than the waves coupled to the tissue. The Doppler signal thus yields the direction of the blood flow which distinguishes arteries from veins, as blood in arteries is flowing away from the heart, and blood in veins is flowing towards the heart. This flow direction of blood can be marked by a color. The direction of the blood flow might be assigned the color red or blue, indicating a flow towards or away from the ultrasound transducer or optical probe. This is why this technique is called Color Doppler (Ultrasound) technique.

A second possibility similar to the first one is to determine the frequency shift of the Doppler signal as a function of time. The result can be used to calculate the blood flow as a function of time. The flow in a vein is rather constant in time, whereas the flow in an artery is pulsating in nature, the frequency of the pulses representing the heart rate. Thus the pulsating or non-pulsating nature of the blood flow can be used to distinguish an artery from a vein.

A third possibility is to carry out a mechanical palpation. If the tissue containing the blood vessel is subjected to a mechanical pressure, veins have the tendency to collapse, whereas arteries do not collapse due to different vessel wall characteristics. Thus the different behaviour of veins and arteries with respect to mechanical pressure can be used to distinguish arteries from veins. The mechanical palpation can be carried out by pushing an imaging probe onto the skin and following the corresponding signal.

A fourth possibility of distinguishing arteries from veins is establishing the oxygen content in the blood which can be measured by an absorption technique. In a first step the blood vessel is subjected to light of a first wavelength which is well absorbed by low-oxygen blood found in veins. In a second step the blood vessel is subjected to light of a second wavelength which is well absorbed by high-oxygen blood found in arteries. Thus measuring and analyzing the absorption of the two wavelengths allows to distinguish arteries from veins.

According to a further preferred embodiment, the needle or cannula is applicable to blood withdrawal and/or drug infusion and/or blood transfusion, and/or catheter insertion and/or dialysis applications. Hence, the invention can be universally applied to various different medical purposes that require insertion of a needle or cannula into a vascular system of a person. Respective cannula insertion means for fixing the needle or cannula are typically realized by making use of a modular concept allowing a quick and secure adaptation of the needle or cannula insertion system to a multitude of different purposes.

These and other aspects of the invention will be apparent from and elucidated with reference to the embodiments described hereafter.

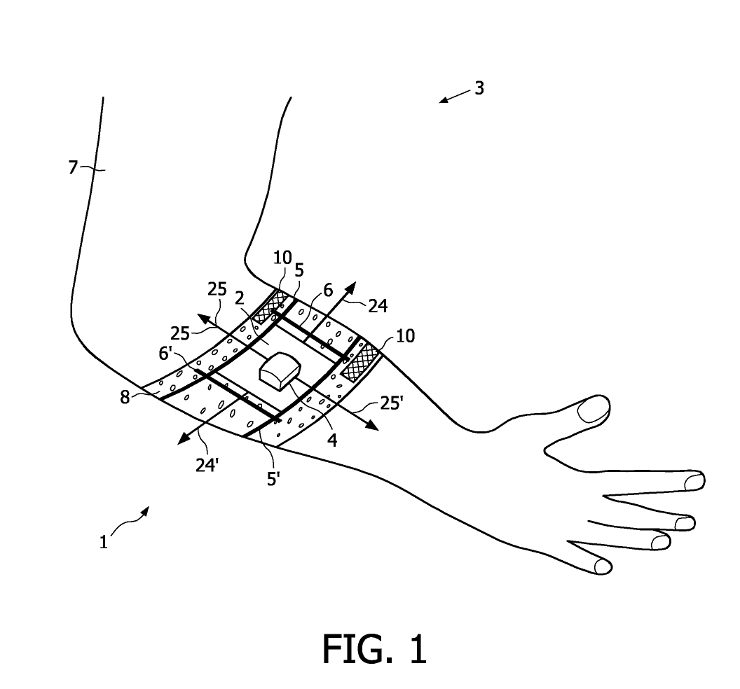

FIG. 1 shows the guiding system attached to a patient's arm,

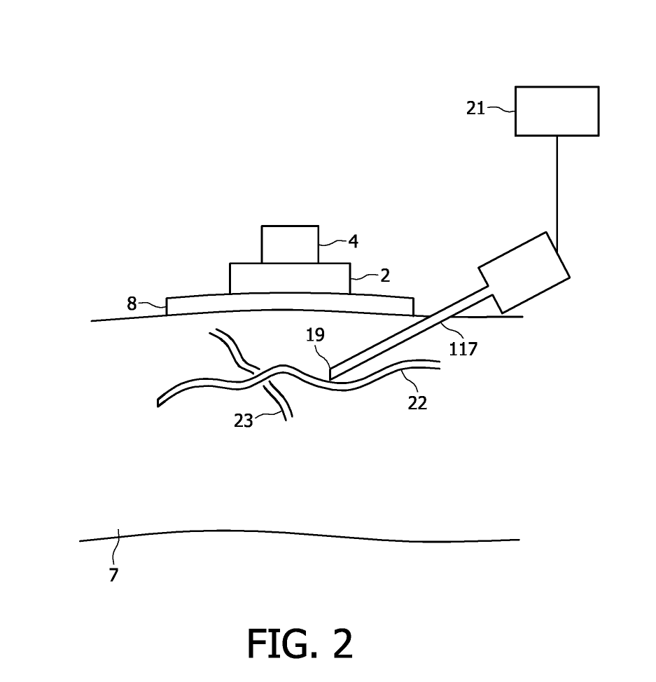

FIG. 2 shows a side view of the guiding system attached to a patient's arm,

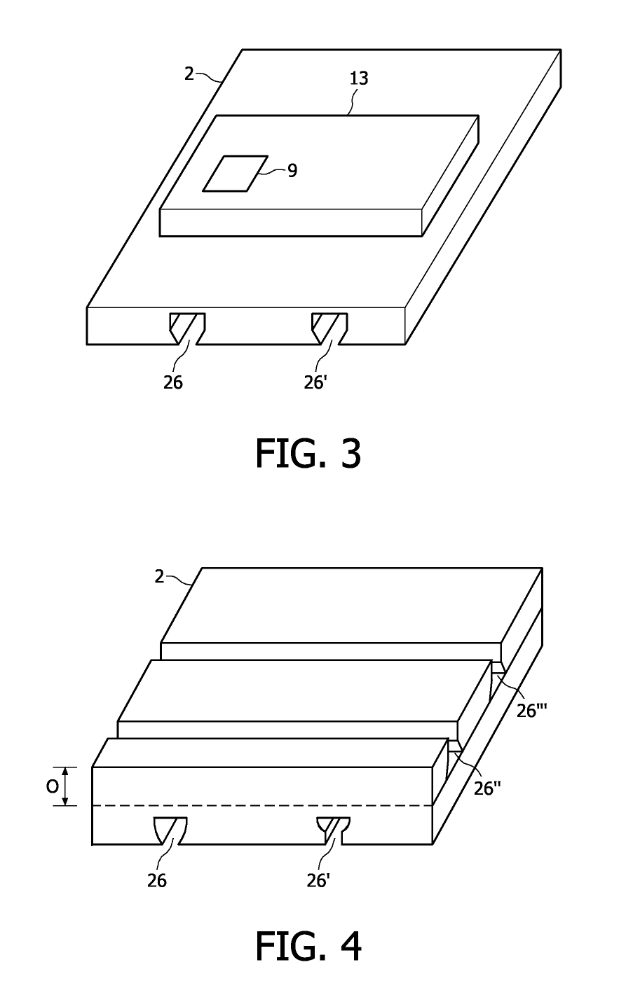

FIG. 3 shows a probe holder allowing a movement in one direction,

FIG. 4 shows a second holder allowing a movement in two directions,

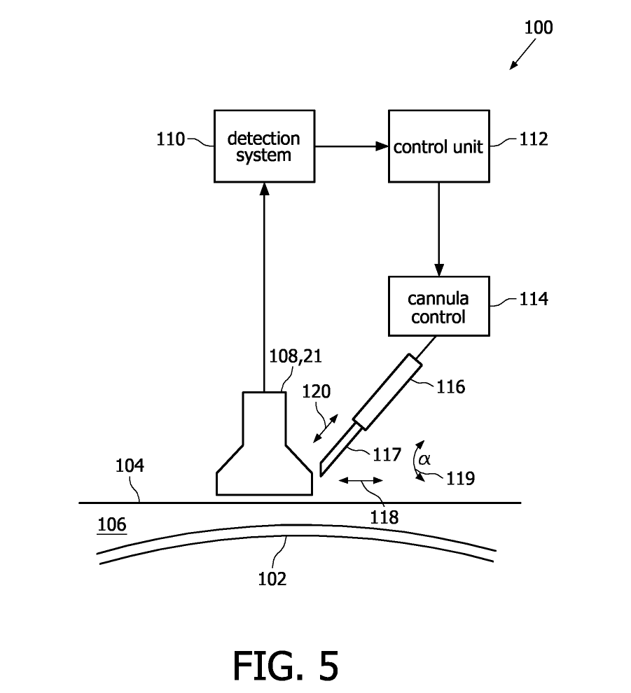

FIG. 5 illustrates a schematic block diagram of the inventive puncture system,

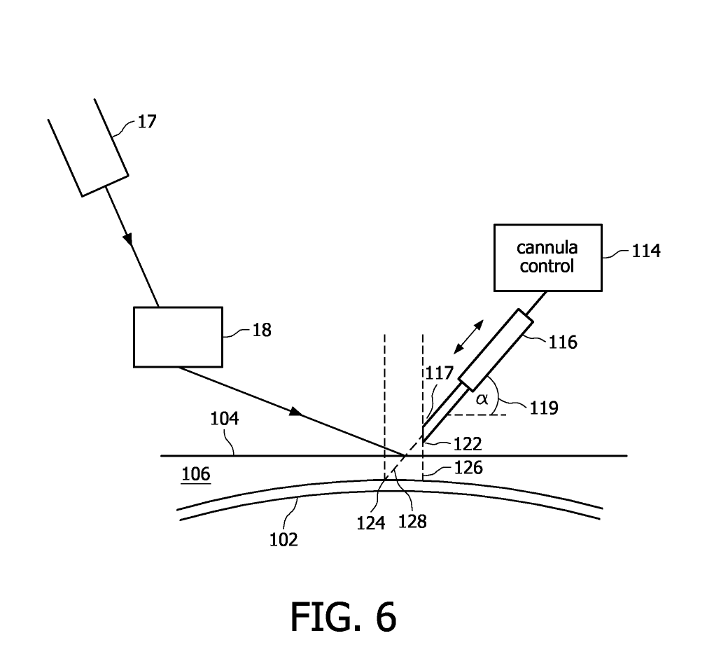

FIG. 6 shows a schematic illustration of a puncture location and of the insertion position determined by the puncture system.

FIG. 1 shows a guiding system 1 according to the invention. The system 1 is wrapped around an arm 7 of a patient 3. It has a strap 8 having a rectangular shape, a width of 15 cm and a length which is adjustable by means of a Velcro fastener 7. By an appropriate use of the Velcro fastener 10 it can be ensured that the strap 8 fits tightly to the arm of the patient 3.

Two flexible rails 5, 5′ are located on top of the strap 8.

The rails 5, 5′ are made of e.g. polypropylene and are spaced approximately 7 cm apart in a parallel configuration. A probe holder 2 is moveably mounted between the rails 5, 5′ and carries a probe 4. The probe 4 is screwed onto the probe holder 2. The probe holder 2 can travel in parallel to the rails 5, 5′ as indicated by the arrows 24 and 24′. The first rails 5,5′ are moveably mounted on second rails 6, 6′, while the alignment of the rails 6, 6′ is perpendicular to the alignment of the rails 5, 5′. As a consequence, the probe holder 2 is moveable parallel as well as vertical to the first rails 5, 5. The choice of two parallel rails in the embodiment of FIG. 1 makes sure that the system is not wobbling around, and that a rotation of the probe holder around the rail is effectively prevented. Furthermore, this stable mounting of the probe holder makes it possible to attach a heavier and/or larger probe securely to the probe holder.

The system according to FIG. 1 is easily attached to the patient, whereby the Velcro fastener 10 allows an individual adjustment of the length of the strap 8 according to the size of the patient or of the body part to which it is attached. Furthermore, it allows a very precise movement of the probe 2 in the directions 24, 24′ and 25, 25′ respectively. To improve this precision even more, the probe holder can be fastened to the rail. Prior to this fastening the position of the rails can be easily changed in order to optimize the measurement position of the probe.

The attachment of the probe holder 2 to the arm 7 of a patient is shown in FIG. 2. The probe holder 2 rests on the strap 8, where the rails are not shown for simplicity. The probe 4 is on top of the probe holder 2 and scans to find blood vessels such as artery 22 or vein 23. As will be discussed below in more detail blood vessel identification means 21 serve to differentiate between veins and arteries. Furthermore, the blood vessel identification means 21 are also adapted to monitor and/or guide the movement of the distal end 19 of cannula 117 into artery 22 or vein 23.

FIG. 3 shows the probe holder 2 in more detail. The probe holder 2 basically comprises a rectangular plate with longitudinal recesses 26, 26′ into which the rails 5, 5′ can be inserted. A movement of the plate relative to the rail can be established in such a way that actuation means 13 comprise a gearwheel (not shown) which has teeth, whereby the teeth engage, as the plate has oblong openings for that purpose, with corresponding openings in the rail. The actuation means 13 also include means 9 for securing a safe position of the probe holder 2 in the rail.

The same approach is used in FIG. 4, which shows a probe holder 2 having recesses 26, 26′ to accommodate a first pair of substantially parallel rails in a first direction. Furthermore, the probe holder 2 has additional recesses 26″ and 26″′ which are arranged with a height offset O in comparison to recesses 26, 26′ as indicated by the double arrow. These additional recesses 26″, 26″′ serve to accommodate a second pair of substantially parallel rails in a second direction being perpendicular to the first direction. A movement in two directions is achieved by gliding of the probe holder 2 along rails 5, 5′ (not shown) accommodated by recesses 26, 26′, and/or by its gliding along rails 6, 6′ (not shown) accommodated by recesses 26″, 26″′.

FIG. 5 shows a schematic block diagram of the puncture system 100 which is mountable on the probe holder 2, where the probe holder 2 is not shown for the sake of simplicity. The puncture system 100 has an acquisition module 108, a detection system 110, a control unit 112, a cannula control 114 as well as a cannula mount 116. The cannula 117 itself can be rigidly attached to the cannula mount 116 that represents fastening means for fixing the cannula and means for moving and aligning the cannula 117 as controlled by the cannula control unit 114. The cannula 117 and the cannula mount 116 can be moved along the insertion direction 120 as well as along direction 118 that is substantially parallel to the surface of the skin 104. In principle direction 118 can be any direction in the plane parallel to the skin surface. Typically, the cannula 117 and the cannula mount 116 are moveable by means of the cannula control 114 in all three spatial directions. Also, the angle α 119 between the insertion direction 120 and the surface of the skin 104 may be arbitrarily modified by means of the cannula control 114 in a way that is determined by means of the detection system 110 and the control unit 112.

FIG. 6 shows an application of the puncture system to a person by means of a cross-sectional illustration of the person's skin 104. Underneath the surface of the skin 104 is a blood vessel 102 that is surrounded by tissue 106. When the puncture system 100 is mounted on the probe holder 2 it is above the skin 104 of the person. The acquisition module 108 is adapted to acquire optical, opto-acoustic or acoustic data from the tissue 106 and the blood vessel 102 that allows to classify at least one blood vessel parameter, such as location of the blood vessel, diameter of the blood vessel, size of the blood vessel, depth underneath the surface of the skin 104, geometry of the blood vessel, blood flow or similar parameters.

Preferably, the acquisition module 108 is realized by means of Ultrasound, Near-infrared imaging, Optical Coherence Tomography, Doppler Ultrasound, Doppler Optical Coherence Tomography or Photo Acoustic techniques that allow to generate a signal providing identification of the blood vessel 102. Signals acquired by the acquisition module 108 are applied to the detection system 110, which in turn generates a signal of the blood vessel 102. Hence, detection system 110 as well as acquisition module 108 are coordinated in a sense that the detection system 110 is suitable for performing signal processing of signals obtained from the acquisition module 108. By making use of optical, opto-acoustic or ultrasound detection, the blood vessel 102 may be precisely located even at an appreciable depth underneath the surface of the skin 104. Additionally, or alternatively, also Doppler techniques may be applied including e.g. Doppler Ultrasound techniques allowing detection of e.g. blood flow in the blood vessel 102. Also, Doppler Optical Coherence Tomography might be correspondingly applied.

Acquisition of location data, geometric data as well as data related to the course of the blood vessel 102, may also be obtained without an imaging of the blood vessel. Therefore, the imaging system 110 does not necessarily have to provide a visual image. Instead, the imaging system 110 may be enabled to directly extract blood vessel parameters from the signals acquired by the acquisition module 108. Hence, extraction of blood vessel parameters may be performed by means of the detection system 110 or by the control unit 112.

The control unit 112 has a processing unit that is enabled to process the data obtained from the detection system 110. Depending on the type of data provided by the detection system 110, the processing unit of the control unit 112 may further process blood vessel parameters in order to extract required blood vessel parameters from a signal of the blood vessel 102. Furthermore, the control unit 112 is enabled to perform a tissue analysis to check whether the tissue in the proximity of the puncture location is suitable for punctuation.

The control unit 112 serves to process the blood vessel parameters in order to find and determine a puncture location of the blood vessel 102 that is ideally suited for an insertion of the cannula 117. In a basic embodiment this puncture location may be determined with respect to location and course of the blood vessel 102. More sophisticated implementations further account for the vessel geometry in the vicinity of an intended puncture location as well as vessel diameter and depth underneath the surface of the skin 104.

Typically, the puncture location may be determined as a result of an optimization procedure taking into account all kinds of blood vessel parameters. For instance, the optimization procedure that is typically performed by means of the processing unit of the control unit 112 may specify, that a puncture location must not be in the vicinity of a branch or junction of a blood vessel 102. Further, a puncture location may require a certain diameter of the blood vessel 102. Also, the puncture location may be determined with respect to a smallest possible depth of the blood vessel 102 underneath the surface of the skin 104. Additionally, the control unit may also determine the insertion direction 120 specifying at what angle α 119 the cannula 117 has to be introduced into the skin 104 and the tissue 106.

Having determined the puncture location, the control unit 112 is further adapted to specify an insertion position for the cannula 117. As can be derived from FIG. 6, the insertion position specifies a position as well as an alignment or direction of the cannula 117 from which the cannula 117 has to be shifted along the insertion direction, i.e. the direction coinciding with the longitudinal direction of the cannula, in order to hit the blood vessel at the determined puncture location with its distal end.

After specifying a puncture location tissue analysis means check whether the tissue 106 surrounding the insertion position 126 is suitable for puncturing. In the alternative, a reverse order is chosen: in a first step the skin surface is analysed, and if this is ok, a blood vessel is determined. The tissue analysis means might be separate means or are provided as an additional functionality of the control unit 112. For the latter case the firmware of the control unit 112 has to be supplemented accordingly. Control unit 112 then needs to analyse the output of the detection system 110 adapted to provide a measurement of the puncture location 124.

Cannulation starts after determining the puncture location 124 and the insertion position 126, whereby the start is either triggered by the operator, or autonomously decided by the puncture system. As soon as the cannula 117 advances towards the blood vessel 102, the acquisition module 108 also acquires position data of the distal end of the cannula 117. Especially, when the cannula 117 already penetrated the skin 104, detection of its distal end allows to control the movement of the cannula 117 through the tissue. As soon as the acquisition module 108 detects that the distal end of the cannula 117 does not properly hit the blood vessel 102, the entire process of cannula insertion may be aborted and the cannula 117 might be withdrawn. In this way, simultaneous acquisition of blood vessel related data and position data of the distal end of the cannula 117 allows to effectively realize a feedback and security mechanism for the autonomous puncture system.

Instead of tracking the end 122 of the needle or cannula it is also possible to monitor and follow the position or movement of the blood vessel 102 during insertion, which is a simpler solution than the one described in the previous paragraph. If it is known where the needle or cannula 117 has to end up and if the insertion parameters have been determined, the location of the blood vessel 102 can be monitored during insertion. The insertion, however, goes wrong if the blood vessel moves.

A particular advantage stems from the fact that the probe holder 2 accommodates an actuation means 13, cf. FIG. 3, and that the above-mentioned puncture system 100 cooperates with it. The actuation means is adapted to operate in response to an output of the puncture system 100, so that the puncture system 100 becomes an automatic blood vessel finder and puncture location finder.

As can be derived from FIG. 6, the puncture system 100 comprises a laser 17 and a projection means 18, the latter comprising a tiltable mirror, to project light onto the puncture location 124. This is helpful for a physician as it visually guides him where to insert a cannula 117 accurately into the patient's body.

Furthermore, the puncture system comprises a blood vessel identification means which is identical to the acquisition module 108, i.e. the acquisition module 108 is adapted to distinguish whether the blood vessel is an artery 22 or a vein 23.

### LIST OF REFERENCE NUMERALS

01 system
02 probe holder
03 patient
04 probe
05 rail
05′ rail
06 rail
06′ rail
07 part of the body/arm
08 strap
09 securing means
10 Velcro fastener
13 actuation means
17 light source
18 projection means
19 distal end
20 control means
21 blood vessel identification means
22 artery
23 vein
24 arrow
24′ arrow
25 arrow
25′ arrow
26 recess
26′ recess
26″ recess
26″′ recess
100 puncture system
102 blood vessel
104 skin
106 tissue
108 acquisition module
110 detection system
112 control unit
114 cannula control
116 cannula mount
117 cannula
118 direction parallel to skin
119 angle
120 insertion direction
122 distal end of cannula
124 puncture location
126 insertion position
128 insertion path

1. A system (1) for guiding a probe holder (2) over the skin of a patient (3) comprising:
a) a probe holder (2) to which a probe (4) is rigidly attachable,
b) at least one flexible rail (5, 5′, 6, 6′), which can be wrapped around at least a part of the body (7) of the patient,
c) whereby the probe holder is moveably mountable or is moveably mounted on said rail.

2. The system according to claim 1, characterized in that the system comprises at least two flexible rails being arranged substantially parallel to each other, and that the probe holder is moveably mountable or is moveably mounted between said rails.

3. The system according to claim 1, characterized in that the probe holder is moveable substantially parallel as well as substantially vertical to the rail.

4. The system according to claim 1, characterized in that the rail is mounted onto a strap (8) which strap can be wrapped around the patient or the patient's body part.

5. The system according to claim 1, characterized in that the system further comprises means (9) for securing the position of the probe holder relative to the rail.

6. The system according to claim 1, characterized in that the system comprises actuation means (13) which are adapted to move the probe holder along the rails.

7. The system according to claim 1, characterized in that the system accommodates a puncture system (100) for inserting a cannula or a needle (117) into a blood vessel (102) of the patient.

8. The system according to claim 7, characterized in that the puncture system comprises
a) location determining means (108) for determining at least one location of the blood vessel, and
b) processing means (110) for determining a puncture location (124) of the blood vessel in response to an output of the location determining means.

9. The system according to claim 7, characterized in that the puncture system comprises
a) a light source (17),
b) projection means (18) associated with the light source, the projection means being adapted to indicate the puncture location and the course of the blood vessel on the skin of the patient by light.

10. The system of claim 8, characterized in that the location determining means are adapted to track the location of the cannula's distal end (19) during insertion of the cannula, the system further comprising control means (20) for controlling the movement of the cannula in response to the tracking of the cannula's distal end.

11. The system of claim 8, characterized in that the location determining means are adapted to track the location and size of the blood vessel during insertion of the cannula and further comprise control means for controlling the movement of the cannula in response to the tracking of the blood vessel parameters.

12. The system of claim 7, characterized in that the puncture system comprises blood vessel identification means (21) for distinguishing an artery (22) from a vein (23).

13. A puncture system according to claim 12, characterized in that the blood vessel identification means are adapted to measure the direction of the blood flow, to analyse the flow characteristics of the blood, to carry out an identification on the basis of mechanical palpation, or is adapted to measure the oxygen content in the blood.
